(19) United States
(12) Patent Application Publication
(10) Pub. No.: US 2023/0204983 A1
(43) Pub. Date: Jun. 29, 2023

(54) BLUE LIGHT FILTERING OPHTHALMIC LENS CROSS-REFERENCE TO RELATED APPLICATIONS

(71) Applicant: YUNG SHENG OPTICAL CO., LTD., TAICHUNG CITY (TW)

(72) Inventors: Kuan Hung Chen, TAICHUNG CITY (TW); Yu-Han Tsao, TAICHUNG CITY (TW); Nien-Chia Lee, TAICHUNG CITY (TW)

(21) Appl. No.: 18/087,044

(22) Filed: Dec. 22, 2022

(30) Foreign Application Priority Data
Dec. 24, 2021 (TW) 110148798

(51) Int. Cl. G02C 7/10; C07D 249/20

(52) U.S. Cl. CPC G02C 7/104; G02C 7/108; C07D 249/20

(57) ABSTRACT

This disclosure provides a blue light filtering ophthalmic lens, which is provided with db* value smaller than or equaled to 2. The ophthalmic lens includes a blue light filtering compound and a base material, wherein db*=(b*)₁−(b*)₀, (b*)₁ is a b* value of the ophthalmic lens and (b*)₀ is a b* value of a control group lens.

### CROSS-REFERENCE TO RELATED APPLICATIONS

This non-provisional application claims priority claim under 35 U.S.C. § 119(a) on Taiwan Patent Application No. 110148798 filed Dec. 24, 2021, the entire contents of which are hereby incorporated by reference.

### BACKGROUND

### Technical Field

This disclosure relates to blue light filtering ophthalmic lenses, in particular to a colorless, transparent or non-yellowing lens for filtering blue light.

### Related Art

With the rapid development of technology, electronic products are more and more important to people, and electronic products have been an important part of our daily life. However, the display panels of most electronic products, such as LCD screens, cell phone screens, etc., need to have a more saturated color rendering in the visible wavelength to satisfy consumers. Therefore, most of the display panels of these products emit significant blue light.

Blue light is generally defined as light with wavelengths between 380 nm and 460 nm, which has a higher energy in visible light spectrum. When using electronic products, blue light can penetrate the human eye and reach the retina. If the retina is exposed to blue light for a long time, the retinal pigment epithelium will be harmed by blue light, resulting in vision loss. Therefore, if users let these electronic products emit blue light to the eyes for a long time, the eyes will suffer irreversible injuries.

In order to reduce or avoid the injury to human eyes caused by blue light, there are many blue light filtering lenses or products available on the market, such as blue light protection glasses, blue light filtering films, etc. Currently, most of the lenses for filtering blue light are made by adding substances with blue light filtering properties to the base material of the lenses, so as to block the blue light from reaching the user's eyes directly. However, when the blue filtering substance filters out the blue light from the visible light, it also causes the lens to have a yellowish appearance. The yellowish color of the product does not look good, causing consumers to be less inclined to buy the yellowish products.

### SUMMARY

An object of this disclosure is to provide a colorless, transparent or non-yellowing lens for filtering blue light.

In order to achieve the above object, This disclosure provides a blue light filtering ophthalmic lens provided with db* value smaller than or equaled to 2, The ophthalmic lens includes a blue light filtering compound and a base material, wherein db*=(b*)₁−(b*)₀, (b*)₁ is a b* value of the ophthalmic lens and (b*)₀ is a b* value of a control group lens.

In order to achieve the above object, This disclosure provides a blue light filtering ophthalmic lens provided with db* value smaller than or equaled to 2 and a blue light filtering rate between 10% to 60%, The ophthalmic lens includes a blue light filtering compound and a base material, wherein db*=(b*)₁−(b*)₀, (b*)₁ is a b* value of the ophthalmic lens and (b*)₀ is a b* value of a control group lens.

The b* value is the value on the blue-yellow coordinate axis in the CIELAB color space.

In at least one example, the blue light filtering compound has a mass percentage concentration of 0.4% to 10%, 1% to 10%, or 5% to 10%.

According to one or more embodiments of this disclosure, the ophthalmic lenses as described above can have a thickness of 0.04 mm to 2.00 mm, 0.04 mm to 1.80 mm, or 0.04 mm to 1.50 mm.

In order to achieve the above object, this disclosure provides a blue light filtering ophthalmic lens provided a thickness of 0.04 mm to 1.5 mm and a blue light filtering rate of 10% to 60%, and the ophthalmic lens includes a base material and a blue light filtering compound of structural formula (A):

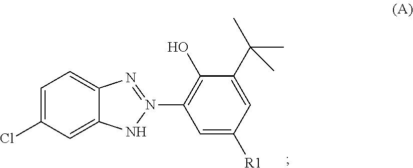

Wherein R1 is hydrogen, C1 to C10 linear alkyl, C1 to C10 branched alkyl, C6 to C15 aryl alkyl, —R2-X, —O—R2-X, or —N—R2-X; R2 is C1 to C10 linear alkylidene, C1 to C10 branched alkylidene, C1 to C10 linear alkylidene containing a hydroxyl substituent, C1 to C10 branched alkylidene containing a hydroxyl substituent, a C1 to C10 linear alkylidene interrupted by an ester group, or C1 to C10 branched alkylidene interrupted by an ester group; wherein X is —OH, —OC(O)R3, —NH2, —NC(O)R3, —NCO, —COOH, or —COOR3; and R3 is linear alkyl from C1 to C10, branched alkyl from C1 to C10, linear alkenyl from C3 to C10, or branched alkenyl from C3 to C10.

In at least one example, wherein the blue light filtering compound has a mass percentage concentration of 0.4% to 10%.

In order to achieve the above object, This disclosure provides a blue light filtering ophthalmic lens which is obtainable by the following steps: mixing a blue light filtering compound and a base material to obtain a mixture solution; wherein the blue light filtering compound has a mass percentage concentration of 0.4% to 10%, and the base material has a mass percentage concentration of 90.0% to 99.6%; adding the mixture solution into a mold; and apply a copolymerization of the mixture solution in the mold to obtain the blue light filtering ophthalmic lens, wherein the blue light filtering ophthalmic lens provided with db* value smaller than or equaled to 2, and db*=(b*)₁−(b*)₀, (b*)₁ is a b* value of the ophthalmic lens and (b*)₀ is a b* value of a control group lens.

In order to achieve the above object, This disclosure provides a blue light filtering ophthalmic lens which is obtainable by the following steps: mixing a blue light filtering compound and a base material to obtain a mixture solution; wherein the blue light filtering compound has a mass percentage concentration of 0.4% to 10%, and the base material has a mass percentage concentration of 90.0% to 99.6%; adding the mixture solution into a mold; and apply a copolymerization of the mixture solution in the mold to obtain the blue light filtering ophthalmic lens, wherein the blue light filtering ophthalmic lens is provided with a blue light filtering rate between 10% to 60%, and the blue light filtering compound is a compound of structural formula (A):

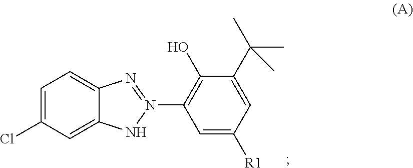

Wherein R1 is hydrogen, C1 to C10 linear alkyl, C1 to C10 branched alkyl, C6 to C15 aryl alkyl, —R2-X, —O—R2-X, or —N—R2-X; R2 is C1 to C10 linear alkylidene, C1 to C10 branched alkylidene, C1 to C10 linear alkylidene containing a hydroxyl substituent, C1 to C10 branched alkylidene containing a hydroxyl substituent, a C1 to C10 linear alkylidene interrupted by an ester group, or C1 to C10 branched alkylidene interrupted by an ester group; X is —OH, —OC(O)R3, —NH2, —NC(O)R3, —NCO, —COOH, or —COOR3; and R3 is linear alkyl from C1 to C10, branched alkyl from C1 to C10, linear alkenyl from C3 to C10, or branched alkenyl from C3 to C10.

When obtaining the blue light filtering ophthalmic lens of this disclosure, the base material having a mass percentage concentration of 90.0% to 99.6% is used.

According to one or more embodiments of this disclosure, the base material as above mentioned is selected form a group consisting of a hydrophilic substance, a polymerization initiator, a cross-linking agent, and a combination thereof.

The hydrophilic substance is selected from a group consisting of 2-hydroxyethyl methacrylate (2-HEMA), methacrylic acid (MAA), acrylic acid (AA), N-vinyl pyrrolidone (NVP), N,N-dimethylacrylamide (DMAA), glycidyl methacrylate (GMA), diethylaminoethyl methacrylate (DEAEMA), and the combination thereof.

The polymerization initiator is a thermal polymerization initiator or a photopolymerization initiator.

The thermal polymerization initiator is selected form a group consisting of azobisisisobutyronitrile (AIBN), azo diisobutyronitrile (ADVN), benzoyl peroxide (BPO), and a combination thereof.

The photopolymerization initiator is phenyl bis(2,4,6-trimethylbenzoyl)-phosphine oxide or 2-hydroxy-2-methyl-1-phenyl-1-propanone.

The cross-linking agent is selected form a group consisting of Ethylene glycol dimethacrylate (EGDMA), triethylene glycol dimethacrylate (TrEGDMA), tetraethylene glycol dimethacrylate (TEGDMA), polyethylene glycol dimethacrylate (PEGDMA), propylene terminated ethylene oxide dimethylsiloxane-ethylene oxide ABA block copolymer, trihydroxymethylpropane trimethacrylate (TMPTMA), and a combination thereof.

In addition, in one embodiment, the base material comprises a hydrophobic substance, and the hydrophobic substance is selected from a group consisting of (3-methacryloyloxy-2-hydroxypropoxy)propylbis(trimethylsiloxy)methyl (SIGMMA), methacryloxypropyltris(trimethylsiloxy)silane (TRIS), polydimethylsiloxane (PDMS), and a combination thereof.

According to one or more embodiments of this disclosure, the blue light filtering compound is a compound of structural formula (A):

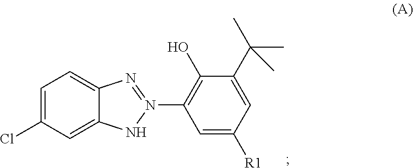

wherein R1 is hydrogen, C1 to C10 linear alkyl, C1 to C10 branched alkyl, C6 to C15 aryl alkyl, —R2-X, —O—R2-X, or —N—R2-X; R2 is C1 to C10 linear alkylidene, C1 to C10 branched alkylidene, C1 to C10 linear alkylidene containing a hydroxyl substituent, C1 to C10 branched alkylidene containing a hydroxyl substituent, a C1 to C10 linear alkylidene interrupted by an ester group, or C1 to C10 branched alkylidene interrupted by an ester group; wherein X is —OH, —OC(O)R3, —NH2, —NC(O)R3, —NCO, —COOH, or —COOR3; and R3 is linear alkyl from C1 to C10, branched alkyl from C1 to C10, linear alkenyl from C3 to C10, or branched alkenyl from C3 to C10.

The ophthalmic lens of this disclosure is preferably a contact lens, for example, a soft contact lens, a rigid contact lens, an embedded ophthalmic lens, or an artificial crystal.

### BRIEF DESCRIPTION OF THE DRAWINGS

This disclosure will become more fully understood from the detailed description given herein below for illustration only, and thus not limitative of this disclosure, wherein:

FIG. 1 shows the light transmittances (T %) of lenses 12 to 16 of each wavelength.

### DETAILED DESCRIPTION

Unless otherwise defined, all technical and scientific terms used herein have the meaning generally understood by person having ordinary skill in the art.

The indefinite article “a”, when used herein, refers to at least one (one or more) number, unless otherwise specified.

This disclosure provides a blue light filtering ophthalmic lens provided with db* value smaller than or equaled to 2. The ophthalmic lens includes a blue light filtering compound and a base material. And db*=(b*)₁−(b*)₀, (b*)₁ is a b* value of the ophthalmic lens and (b*)₀ is a b* value of a control group lens.

In at least one embodiment, this disclosure provides a blue light filtering ophthalmic lens provided with db* value smaller than or equaled to 2 and a blue light filtering rate between 10% to 60%. The ophthalmic lens includes a blue light filtering compound and a base material. And db*=(b*)₁−(b*)₀, (b*)₁ is a b* value of the ophthalmic lens and (b*)₀ is a b* value of a control group lens.

The control group lens is a lens with no blue light filtering compound added.

The b* value is the value on the blue-yellow coordinate axis in the CIELAB color space. The relevant definition of CIELAB color space can be found in published literatures, e.g., U.S. Pat. No. 5,751,845 or US 2019/0151161.

In at least one example, the blue light filtering compound has a mass percentage concentration of 0.4% to 10%.

According to one or more embodiments of this disclosure, the blue light filtering compound has a mass percentage concentration of 0.4% to 10%, 1% to 10%, 1.5% to 10%, 2% to 10%, 2.5% to 10%, 3% to 10%, 3.5% to 10%, 4% to 10%, 4.5% to 10%, or 5% to 10%.

According to one or more embodiments of this disclosure, the blue light filtering ophthalmic lens as above mentioned is provided with a thickness of 0.04 mm to 2.00 mm, 0.04 mm to 1.95 mm, 0.04 mm to 1.90 mm, 0.04 mm to 1.85 mm, 0.04 mm to 1.80 mm, 0.04 mm to 1.75 mm, 0.04 mm to 1.70 mm, 0.04 mm to 1.65 mm, 0.04 mm to 1.60 mm, 0.04 mm to 1.55 mm, or 0.04 mm to 1.50 mm.

The blue light filtering ophthalmic lens as above mentioned is provided with a blue light filtering rate between 10% to 60%, 20% to 60%, 30% to 60%, 40% to 60%, or 50% to 60%.

In at least one embodiment, this disclosure provides a blue light filtering ophthalmic lens, provided a thickness of 0.04 mm to 1.5 mm and a blue light filtering rate of 10% to 60%, and the ophthalmic lens includes a base material and a blue light filtering compound of structural formula (A):

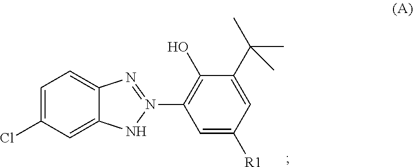

Wherein R1 is hydrogen, C1 to C10 linear alkyl, C1 to C10 branched alkyl, C6 to C15 aryl alkyl, —R2-X, —O—R2-X, or —N—R2-X; R2 is C1 to C10 linear alkylidene, C1 to C10 branched alkylidene, C1 to C10 linear alkylidene containing a hydroxyl substituent, C1 to C10 branched alkylidene containing a hydroxyl substituent, a C1 to C10 linear alkylidene interrupted by an ester group, or C1 to C10 branched alkylidene interrupted by an ester group; wherein X is —OH, —OC(O)R3, —NH2, —NC(O)R3, —NCO, —COOH, or —COOR3; and R3 is linear alkyl from C1 to C10, branched alkyl from C1 to C10, linear alkenyl from C3 to C10, or branched alkenyl from C3 to C10.

In at least one example, the blue light filtering compound has a mass percentage concentration of 0.4% to 10%.

In at least one embodiment, This disclosure provides a blue light filtering ophthalmic lens, which is obtainable by: mixing a blue light filtering compound and a base material to obtain a mixture solution; wherein the blue light filtering compound has a mass percentage concentration of 0.4% to 10%, and the base material has a mass percentage concentration of 90.0% to 99.6%; adding the mixture solution into a mold; and apply a copolymerization of the mixture solution in the mold to obtain the blue light filtering ophthalmic lens, wherein the blue light filtering ophthalmic lens provided with db* value smaller than or equaled to 2, and db*=(b*)₁−(b*)₀, (b*)₁ is a b* value of the ophthalmic lens and (b*)₀ is a b* value of a control group lens.

For example, the control group lens is a lens with no blue light filtering compound added. The b* value is the value on the blue-yellow coordinate axis in the CIELAB color space.

In at least one embodiment, This disclosure provides a blue light filtering ophthalmic lens, which is obtainable by: mixing a blue light filtering compound and a base material to obtain a mixture solution; wherein the blue light filtering compound has a mass percentage concentration of 0.4% to 10%, and the base material has a mass percentage concentration of 90.0% to 99.6%; adding the mixture solution into a mold; and apply a copolymerization of the mixture solution in the mold to obtain the blue light filtering ophthalmic lens, wherein the blue light filtering ophthalmic lens is provided with a blue light filtering rate between 10% to 60%, and the blue light filtering compound is a compound of structural formula (A):

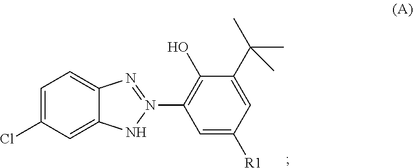

Wherein R1 is hydrogen, C1 to C10 linear alkyl, C1 to C10 branched alkyl, C6 to C15 aryl alkyl, —R2-X, —O—R2-X, or —N—R2-X; R2 is C1 to C10 linear alkylidene, C1 to C10 branched alkylidene, C1 to C10 linear alkylidene containing a hydroxyl substituent, C1 to C10 branched alkylidene containing a hydroxyl substituent, a C1 to C10 linear alkylidene interrupted by an ester group, or C1 to C10 branched alkylidene interrupted by an ester group; X is —OH, —OC(O)R3, —NH2, —NC(O)R3, —NCO, —COOH, or —COOR3; and R3 is linear alkyl from C1 to C10, branched alkyl from C1 to C10, linear alkenyl from C3 to C10, or branched alkenyl from C3 to C10.

When obtaining the blue light filtering ophthalmic lens of this disclosure, the base material having a mass percentage concentration of 90.0% to 99.6% is used.

According to one or more embodiments of this disclosure, the base material as above mentioned is selected form a group consisting of a hydrophilic substance, a hydrophobic substance, a polymerization initiator, a cross-linking agent, and a combination thereof.

The hydrophilic substance is selected from a group consisting of 2-hydroxyethyl methacrylate (2-HEMA), methacrylic acid (MAA), acrylic acid (AA), N-vinyl-2-pyrrolidone (NVP), N,N-dimethylacrylamide (DMAA), glycidyl methacrylate (GMA), diethylaminoethyl methacrylate (DEAEMA), other equivalent compound, and the combination thereof.

The polymerization initiator is a thermal polymerization initiator or a photopolymerization initiator. It should be noted that the thermal polymerization initiator is capable of initiating a chemical reaction (polymerization) between the base material and the blue light filtering compound when heated, and the photopolymerization initiator is capable of initiating a chemical reaction (polymerization) between the base material and the blue light filtering compound when irradiated by light.

The thermal polymerization initiator is selected form a group consisting of azobisisisobutyronitrile (AIBN), azo diisobutyronitrile (ADVN), benzoyl peroxide (BPO), and a combination thereof.

The photopolymerization initiator is phenyl bis(2,4,6-trimethylbenzoyl)-phosphine oxide or 2-hydroxy-2-methyl-1-phenyl-1-propanone.

Preferably, the base material further includes a cross-linking agent, the cross-linking agent is selected form a group consisting of Ethylene glycol dimethacrylate (EGDMA), triethylene glycol dimethacrylate (TrEGDMA), tetraethylene glycol dimethacrylate (TEGDMA), polyethylene glycol dimethacrylate (PEGDMA), propylene terminated ethylene oxide dimethylsiloxane-ethylene oxide ABA block copolymer, trihydroxymethylpropane trimethacrylate (TMPTMA), other equivalent compound, and a combination thereof.

In addition, in one embodiment, the base material further includes a hydrophobic substance. The hydrophobic substance is selected from a group consisting of (3-methacryloyloxy-2-hydroxypropoxy)propylbis(trimethylsiloxy)methyl (SIGMMA), methacryloxypropyltris(trimethylsiloxy)silane (TRIS), polydimethylsiloxane (PDMS), other equivalent compound, and a combination thereof.

For example, the base material may include a hydrophobic substance, include a hydrophobic substance, or simultaneously include the hydrophilic substance and the hydrophobic substance.

According to one or more embodiments of this disclosure, the blue light filtering compound as above mentioned is a compound of structural formula (A):

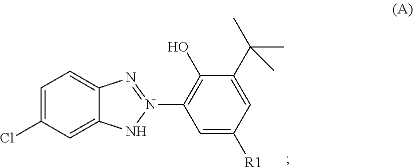

wherein R1 is hydrogen, C1 to C10 linear alkyl, C1 to C10 branched alkyl, C6 to C15 aryl alkyl, —R2-X, —O—R2-X, or —N—R2-X; R2 is C1 to C10 linear alkylidene, C1 to C10 branched alkylidene, C1 to C10 linear alkylidene containing a hydroxyl substituent, C1 to C10 branched alkylidene containing a hydroxyl substituent, a C1 to C10 linear alkylidene interrupted by an ester group, or C1 to C10 branched alkylidene interrupted by an ester group; wherein X is —OH, —OC(O)R3, —NH2, —NC(O)R3, —NCO, —COOH, or —COOR3; and R3 is linear alkyl from C1 to C10, branched alkyl from C1 to C10, linear alkenyl from C3 to C10, or branched alkenyl from C3 to C10.

According to one or more embodiments of this disclosure, the blue light filtering compound is a compound of structural formula (A):

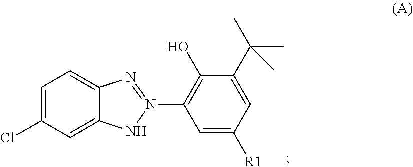

Wherein R1 is hydrogen, C1 to C10 linear alkyl, C1 to C10 branched alkyl, C6 to C15 aryl alkyl, —R2-X, —O—R2-X, or —N—R2-X; R2 is C1 to C10 linear alkylidene, C1 to C10 branched alkylidene, C1 to C10 linear alkylidene containing a hydroxyl substituent, C1 to C10 branched alkylidene containing a hydroxyl substituent, a C1 to C10 linear alkylidene interrupted by an ester group, or C1 to C10 branched alkylidene interrupted by an ester group; wherein X is —OH, —OC(O)R3, —NH2, —NC(O)R3, —NCO, —COOH, or —COOR3; and R3 is linear alkyl from C1 to C10, branched alkyl from C1 to C10, linear alkenyl from C3 to C10, or branched alkenyl from C3 to C10.

In at least one example, blue light filtering ophthalmic lens provided with a db* value smaller than or equaled to 2 is obtainable by: mixing a blue light filtering compound and a base material to obtain a mixture solution; wherein the blue light filtering compound has a mass percentage concentration of 1% to 10%, and the base material has a mass percentage concentration of 90% to 99%; adding the mixture solution into a mold; and apply a copolymerization of the mixture solution in the mold to obtain the blue light filtering ophthalmic lens.

The blue light filtering ophthalmic lens as obtained is provided with a thickness of 0.04 mm to 2.00 mm, 0.04 mm to 1.95 mm, 0.04 mm to 1.90 mm, 0.04 mm to 1.85 mm, 0.04 mm to 1.80 mm, 0.04 mm to 1.75 mm, 0.04 mm to 1.70 mm, 0.04 mm to 1.65 mm, 0.04 mm to 1.60 mm, 0.04 mm to 1.55 mm, or 0.04 mm to 1.50 mm.

In addition, in one embodiment, the blue light filtering ophthalmic lens as obtained is provided with a blue light filtering rate between 10% to 60%, 20% to 60%, 30% to 60%, 40% to 60%, or 50% to 60%.

According to one or more embodiments of this disclosure, the blue light filtering compound used to obtain the blue light filtering compound is a compound of structural formula (A):

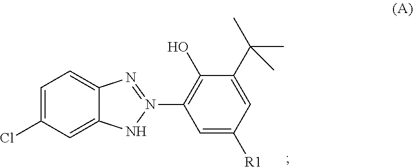

Wherein R1 is hydrogen, C1 to C10 linear alkyl, C1 to C10 branched alkyl, C6 to C15 aryl alkyl, —R2-X, —O—R2-X, or —N—R2-X; R2 is C1 to C10 linear alkylidene, C1 to C10 branched alkylidene, C1 to C10 linear alkylidene containing a hydroxyl substituent, C1 to C10 branched alkylidene containing a hydroxyl substituent, a C1 to C10 linear alkylidene interrupted by an ester group, or C1 to C10 branched alkylidene interrupted by an ester group; wherein X is —OH, —OC(O)R3, —NH2, —NC(O)R3, —NCO, —COOH, or —COOR3; and R3 is linear alkyl from C1 to C10, branched alkyl from C1 to C10, linear alkenyl from C3 to C10, or branched alkenyl from C3 to C10.

The blue light filtering compound in this disclosure can be a compound of structural formula (A1):

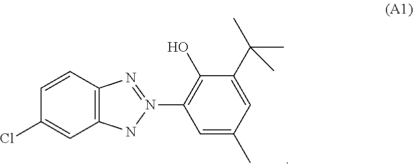

The blue light filtering compound in this disclosure can be a compound of structural formula (A2):

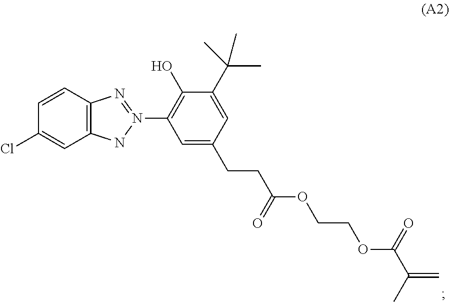

The blue light filtering compound includes a unsaturated vinyl cluster.

The ophthalmic lens of the this disclosure is preferably a contact lens, for example, a soft contact lens, a rigid contact lens, an embedded ophthalmic lens or an artificial crystal.

The following examples are used as further illustrations of this disclosure, and the examples are illustrative and not limiting.

### Example 1: Preparation of Ophthalmic Lenses with Different Raw Materials and Raw Material Ratios

According to the raw materials and their weight ratios shown in Table 1 below, lenses 1 to 11 were prepared.

TABLE 1
Raw material weight ratio table

| Percentage (Wt %) | | Lens 1 | Lens 2 | Lens 3 | Lens 4 | Lens 5 | Lens 6 | Lens 7 | Lens 8 | Lens 9 | Lens 10 | Lens 11 |
|---|---|---|---|---|---|---|---|---|---|---|---|---|
| Molding | HEMA | 99.60 | 99.20 | 98.60 | 96.60 | 94.60 | 89.60 | 99.59 | 99.58 | 99.56 | 99.52 | 99.50 |
| material | EGDMA | 0.20 | 0.20 | 0.20 | 0.20 | 0.20 | 0.20 | 0.20 | 0.20 | 0.20 | 0.20 | 0.20 |
| | AIBN | 0.20 | 0.20 | 0.20 | 0.20 | 0.20 | 0.20 | 0.20 | 0.20 | 0.20 | 0.20 | 0.20 |
| Blue light | Yellow Dye | — | — | — | — | — | — | 0.01 | 0.02 | 0.04 | 0.08 | 0.10 |
| filtering | Compound of | — | 0.40 | 1.00 | 2.00 | 5.00 | 10.00 | — | — | — | — | — |
| compound | structure (A) | | | | | | | | | | | |

| | | | | | | | | | | | |
|---|---|---|---|---|---|---|---|---|---|---|---|
| Total (Wt %) | 100.00 | 100.00 | 100.00 | 100.00 | 100.00 | 100.00 | 100.00 | 100.00 | 100.00 | 100.00 | 100.00 |

Wherein 2-HEMA is 2-hydroxyethyl methacrylate, EGDMA is ethylene glycol dimethacrylate, and AIBN is azo diisobutyronitrile.

Take lens 2 for example, the raw materials prepared were 0.40% by weight of the compound having structural formula (A), 99.20% by weight of HEMA, 0.20% by weight of EGDMA, and 0.20% by weight of AIBN, The prepared materials are filtered and mixed well and placed in a mold. And then, the materials were heated or irradiated tp apply a copolymerization of the material in the mold. After the reaction is complete, the lens is stripped out of the mold and used for subsequent analysis.

### Example 2: Blue Light Filtering Capability Analysis

The average blue light transmittance (%) in the wavelength range of 380-460 nm for the lenses 1 to 11 prepared in Example 1 was measured using a UV-Vis spectrophotometer (e.g., an Agilent Cary 60 spectrophotometer), respectively. And then respective blue light filtering rate (%) is calculated and used as a parameter to evaluate the blue light filtering capability of the lens.

First, the lens to be tested is soaked in standard salt water solution for at least 30 minutes. Carefully remove the 1 cm optical diameter quartz cuvette and wash it 3 times with pure water and then 3 times with standard saltwater solution. Wipe the outside of the quartz cuvette with a lens tissue and check the surface of the quartz cuvette for dirt or fingerprints. If there is dirt or fingerprints, repeat the washing procedure until clean, or replace the quartz cuvette. Place the lens carefully into the quartz cuvette and cut the lens into a suitable square size if necessary. Start the spectrophotometer and necessary software. The wavelength to be examined to obtain the blue light transmittance of the lens is in the blue light wavelength range of 380 nm to 460 nm. The measurement range is set from 380 nm to 460 nm, and the spectral scanning band width is 1 nm. Add eight-tenths of standard saltwater solution to the quartz cuvette and perform blank calibration on the set spectrophotometer. Hold the quartz cuvette, place the lens carefully, and make sure the center of the lens is in the position where the light source of the spectrophotometer passes through. Measure the light transmittance (T %) of the lens at wavelengths from 380 nm to 460 nm and store the measurement results. Next, the following equations 1 and 2 are used to calculate the corresponding blue light filtering capacity values:

$$Average blue light transmittence ( T \% ) = \frac{{\sum }_{k = 380 nm}^{460 nm} τ ( λ )}{( 460 - 380 ) + 1} \qquad ( 1 )$$

$$Blue Light Filtering Rate ( \% ) = 100 - Blue Light Transmittance ( \% ) \qquad ( 2 )$$

The measurement results of lenses 1 to 11 and the calculated values are recorded in Table 2 below.

TABLE 2
Blue light filtering capacity value

| Lens | 1 | 2 | 3 | 4 | 5 | 6 | 7 | 8 | 9 | 10 | 11 |
|---|---|---|---|---|---|---|---|---|---|---|---|
| Average blue light | 99.6 | 89.7 | 78.4 | 72.4 | 59.2 | 43.6 | 93.9 | 91.8 | 87.9 | 79.8 | 77.7 |
| transmittence (%) | | | | | | | | | | | |
| blue light filtering | 0.4 | 10.3 | 21.6 | 27.6 | 40.8 | 60.1 | 6.1 | 8.2 | 12.1 | 20.2 | 22.3 |
| rate (%) | | | | | | | | | | | |

As shown in Table 2, Without adding any blue light filtering substance, lens 1 still has a slight blue light filtering rate (about 0.4%).

In the case of adding a yellow dye as a blue light filtering substance, adding a small amount of yellow dye (about 0.1%) will give the lens a yellow appearance and will not satisfy the purpose of this disclosure. Therefore, we need to reduce the proportion of yellow dye in lenses 7 to 11 (see Table 1) to test whether lenses 7 to 11 still have the desired blue filtering effect. The test results show that using 0.01 to 0.1% yellow dye can achieve 6 to 22% blue light filtering rate.

Generally speaking, the blue light filtering rate of the lens must be at least 10% before the lens is considered to have the ability of filtering blue light.

Among the lenses using yellow dye, the blue light filtering rate of lenses 7 and 8 is less than 10%, and the blue light filtering rate of lenses 9 to 11 is greater than 10%. The blue light filtering rate of lenses 2 to 6 with the compound of structure (A) is about 10 to about 60%.

### Example 3: Transparency Analysis

Standard calibration is first performed on a colorimeter (e.g., colorimeter model MSEZ-40005). After completing the calibration, align the lens set of the colorimeter with a control group lens (lens 1 prepared in Example 1) and then press the measurement button. The colorimeter will display the absolute color values for this control group lens: L*, a*, b*.

Next, Align the lens of the colorimeter to an experimental group lens (one of the lenses 2 to 11 prepared in Example 1) to repeat the above test. The colorimeter will display the absolute color values for this experimental group lens: L*, a*, b*.

According to the positive or negative value of the displayed value of the color difference, we can conclude what kind of color difference there is between the experimental group and the control group lenses. wherein: L* represents black and white, a positive value of L* means that the experimental group is whiter than the control group, and a negative value of L* means that the experimental group is darker; a* represents red and green, a positive value of a* means that the experimental group is more red than the control group, and a negative value means that the experimental group is more green. b* represents yellow and blue, a positive value of b* means that the experimental group is more yellow than the control group, and a negative value means that the experimental group is more blue.

This disclosure reveals that whether the appearance of the lens is yellowish can be determined by the db* value calculated from the following equation 3:

(db*)experimental group lens=(b*)experimental group lens−(b*)control group lens (3)

The measured and calculated values for lenses 1 to 11, and the comparison of these values with the appeared color of the lenses and the blue light filtering rates are shown in Table 3 below.

TABLE 3
Chromatic aberration value/appeared color/blue light filtering rate

| | | | | | | Blu light |
|---|---|---|---|---|---|---|
| | | | | | Appeared color | filtering |
| Lens | L* | a* | b* | db* | (observed visually) | rate (%) |
| 1 | 32.10 | −0.02 | 0.00 | — | transparent and colorless | 0.40 |
| 2 | 30.11 | −0.06 | 0.03 | 0.03 | transparent and colorless | 10.3 |
| 3 | 30.11 | −0.14 | 0.08 | 0.08 | transparent and colorless | 21.60 |
| 4 | 29.86 | −0.28 | 0.24 | 0.24 | transparent and colorless | 27.60 |
| 5 | 30.66 | −0.35 | 0.44 | 0.44 | transparent and colorless | 40.80 |
| 6 | 30.44 | −0.60 | 0.87 | 0.87 | transparent and colorless | 60.10 |
| 7 | 30.80 | −0.40 | 0.44 | 0.44 | transparent and colorless | 6.10 |
| 8 | 32.03 | −0.70 | 2.01 | 2.01 | transparent and colorless | 8.20 |
| 9 | 31.76 | −0.80 | 2.53 | 2.53 | Yellow | 12.10 |
| 10 | 31.23 | −1.01 | 3.04 | 3.04 | Yellow | 20.20 |
| 11 | 31.54 | −1.65 | 5.59 | 5.59 | Yellow | 22.30 |

Lens 1 is prepared without any added blue light filtering substance. Therefore, the appearance of lens 1 appears transparent and colorless when observed visually, and its b* value representing the yellow-green information is 0.00. Lens 1 is used as a control group lens.

Lenses 7 to 11 are lenses prepared with yellow dye. Although lenses 7 to 8 have a transparent and colorless appearance, the blue light filtering rate of lenses 7 to 8 is less than 10% (see Table 2), which makes lenses 7 to 8 not have the required blue light filtering ability. Although the blue light filtering rate of lenses 9 to 11 is greater than 10% (see Table 2), the appearance of lenses 9 to 11 is yellowish when observed visually, as shown in Table 3.

Lenses 2 to 6 are lenses made from compounds with structural formula (A). The appearance of lenses 2 to 6 appears transparent and colorless when observed visually, and the db* values of lenses 2 to 6 are between 0.08 and 0.87.

### Example 4: Other Examples

Table 4 below illustrates the relevant parameters of lenses 12 to 16 for other examples of this disclosure.

TABLE 4
relevant parameters of lenses 12 to 16

| Lens | 12 | 13 | 14 | 15 | 16 |
|---|---|---|---|---|---|
| Thickness(mm) | 0.04 | 1.5 | 2.0 | 1.5 | 2.0 |
| blue light filtering | 0.4% | 0.4% | 0.4% | 10% | 10% |
| substance (Wt %) | | | | | |
| db* | 0.03 | 0.3 | 0.49 | 0.57 | 0.91 |
| Appeared color | transparent | transparent | transparent | transparent | transparent |
| (observed visually) | and colorless | and colorless | and colorless | and colorless | and colorless |
| Average light | 89.7 | 63.9 | 52.5 | 45.8 | 39.0 |
| transmittance | | | | | |
| (T %) of 380 nm | | | | | |
| to 460 (T %) | | | | | |
| blue light filtering | 10.3 | 36.1 | 47.5 | 54.2 | 61.0 |
| rate (%) | | | | | |

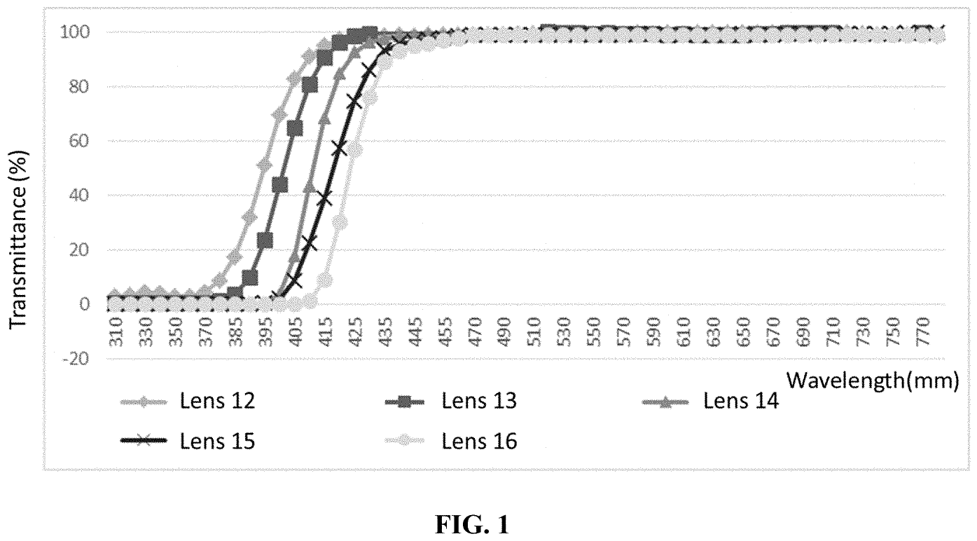

Moreover, FIG. 1 shows the light transmittance (T %) of lenses 12 to 16 of each wavelength.

What is claimed is:

1. A blue light filtering ophthalmic lens provided with db* value smaller than or equaled to 2, comprising a blue light filtering compound and a base material, wherein db*=(b*)₁−(b*)₀, (b*)₁ is a b* value of the ophthalmic lens and (b*)₀ is a b* value of a control group lens.

2. The blue light filtering ophthalmic lens as claimed in claim 1, wherein the blue light filtering ophthalmic lens is a blue light filtering rate between 10% to 60%.

3. The blue light filtering ophthalmic lens as claimed in claim 1, wherein the blue light filtering ophthalmic lens is provided with a thickness of 0.04 mm to 2.00 mm.

4. The blue light filtering ophthalmic lens as claimed in claim 1, wherein the blue light filtering compound has a mass percentage concentration of 0.4% to 10%.

5. The blue light filtering ophthalmic lens as claimed in claim 2, wherein the blue light filtering ophthalmic lens is provided with a thickness of 0.04 mm to 2.00 mm.

6. The blue light filtering ophthalmic lens as claimed in claim 2, wherein the blue light filtering compound has a mass percentage concentration of 0.4% to 10%.

7. The blue light filtering ophthalmic lens as claimed in claim 1, wherein the blue light filtering ophthalmic lens is a soft contact lens, a rigid contact lens, an embedded ophthalmic lens, or an artificial crystal.

8. The blue light filtering ophthalmic lens as claimed in claim 1, wherein the base material is selected form a group consisting of a hydrophilic substance, a polymerization initiator, a cross-linking agent, and a combination thereof.

9. The blue light filtering ophthalmic lens as claimed in claim 8, wherein the polymerization initiator is a thermal polymerization initiator or a photopolymerization initiator.

10. The blue light filtering ophthalmic lens as claimed in claim 9, wherein the thermal polymerization initiator is selected form a group consisting of azobisisisobutyronitrile (AIBN), azo diisobutyronitrile (ADVN), benzoyl peroxide (BPO), and a combination thereof.

11. The blue light filtering ophthalmic lens as claimed in claim 9, wherein the photopolymerization initiator is phenyl bis(2,4,6-trimethylbenzoyl)-phosphine oxide or 2-hydroxy-2-methyl-1-phenyl-1-propanone.

12. The blue light filtering ophthalmic lens as claimed in claim 8, wherein the cross-linking agent is selected form a group consisting of Ethylene glycol dimethacrylate (EGDMA), triethylene glycol dimethacrylate (TrEGDMA), tetraethylene glycol dimethacrylate (TEGDMA), polyethylene glycol dimethacrylate (PEGDMA), propylene terminated ethylene oxide dimethylsiloxane-ethylene oxide ABA block copolymer, trihydroxymethylpropane trimethacrylate (TMPTMA), and a combination thereof.

13. The blue light filtering ophthalmic lens as claimed in claim 8, wherein the hydrophilic substance is selected from a group consisting of 2-hydroxyethyl methacrylate (2-HEMA), methacrylic acid (MAA), acrylic acid (AA), N-vinyl-2-pyrrolidone (NVP), N,N-dimethylacrylamide (DMAA), glycidyl methacrylate (GMA), diethylaminoethyl methacrylate (DEAEMA), and the combination thereof.

14. The blue light filtering ophthalmic lens as claimed in claim 1, wherein the blue light filtering compound is a compound of structural formula (A): 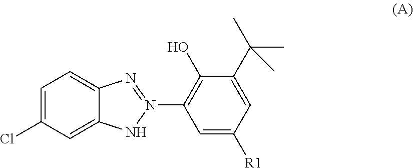
wherein R1 is hydrogen, C1 to C10 linear alkyl, C1 to C10 branched alkyl, C6 to C15 aryl alkyl, —R2-X, —O—R2-X, or —N—R2-X; R2 is C1 to C10 linear alkylidene, C1 to C10 branched alkylidene, C1 to C10 linear alkylidene containing a hydroxyl substituent, C1 to C10 branched alkylidene containing a hydroxyl substituent, a C1 to C10 linear alkylidene interrupted by an ester group, or C1 to C10 branched alkylidene interrupted by an ester group; wherein X is —OH, —OC(O)R3, —NH2, —NC(O)R3, —NCO, —COOH, or —COOR3; R3 is linear alkyl from C1 to C10, branched alkyl from C1 to C10, linear alkenyl from C3 to C10, or branched alkenyl from C3 to C10.

15. A blue light filtering ophthalmic lens provided with a thickness of 0.04 mm to 1.5 mm and a blue light filtering rate of 10% to 60%, comprising a blue light filtering compound of structural formula (A): 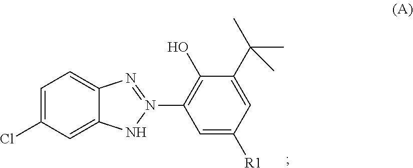
wherein R1 is hydrogen, C1 to C10 linear alkyl, C1 to C10 branched alkyl, C6 to C15 aryl alkyl, —R2-X, —O—R2-X, or —N—R2-X; wherein R2 is C1 to C10 linear alkylidene, C1 to C10 branched alkylidene, C1 to C10 linear alkylidene containing a hydroxyl substituent, C1 to C10 branched alkylidene containing a hydroxyl substituent, a C1 to C10 linear alkylidene interrupted by an ester group, or C1 to C10 branched alkylidene interrupted by an ester group; wherein X is —OH, —OC(O)R3, —NH2, —NC(O)R3, —NCO, —COOH, or —COOR3; wherein R3 is linear alkyl from C1 to C10, branched alkyl from C1 to C10, linear alkenyl from C3 to C10, or branched alkenyl from C3 to C10.

16. The blue light filtering ophthalmic lens as claimed in claim 15, wherein the blue light filtering compound has a mass percentage concentration of 0.4% to 10%.

17. The blue light filtering ophthalmic lens as claimed in claim 16, wherein the base material has a mass percentage concentration of 90.0% to 99.6%.

18. A method for obtaining a blue light filtering ophthalmic lens, comprising steps of:
mixing a blue light filtering compound and a base material to obtain a mixture solution; wherein the blue light filtering compound has a mass percentage concentration of 0.4% to 10%, and the base material has a mass percentage concentration of 90.0% to 99.6%;
adding the mixture solution into a mold; and
apply a copolymerization of the mixture solution in the mold to obtain the blue light filtering ophthalmic lens, wherein the blue light filtering ophthalmic lens provided with db* value smaller than or equaled to 2, db*=(b*)₁−(b*)₀, (b*)₁ is a b* value of the ophthalmic lens and (b*)₀ is a b* value of a control group lens.

19. The method as claimed in claim 18, wherein:
the blue light filtering compound is a compound of structural formula (A): 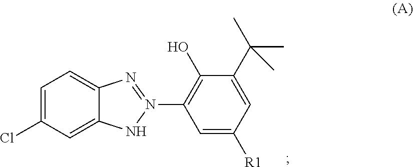
wherein R1 is hydrogen, C1 to C10 linear alkyl, C1 to C10 branched alkyl, C6 to C15 aryl alkyl, —R2-X, —O—R2-X, or —N—R2-X; R2 is C1 to C10 linear alkylidene, C1 to C10 branched alkylidene, C1 to C10 linear alkylidene containing a hydroxyl substituent, C1 to C10 branched alkylidene containing a hydroxyl substituent, a C1 to C10 linear alkylidene interrupted by an ester group, or C1 to C10 branched alkylidene interrupted by an ester group; wherein X is —OH, —OC(O)R3, —NH2, —NC(O)R3, —NCO, —COOH, or —COOR3; and R3 is linear alkyl from C1 to C10, branched alkyl from C1 to C10, linear alkenyl from C3 to C10, or branched alkenyl from C3 to C10.

20. The blue light filtering ophthalmic lens as claimed in claim 18, wherein the base material comprises a hydrophobic substance, and the hydrophobic substance is selected from a group consisting of (3-methacryloyloxy-2-hydroxypropoxy)propylbis(trimethylsiloxy)methyl (SIGMMA), methacryloxypropyltris (trimethylsiloxy)silane (TRIS), polydimethylsiloxane (PDMS), and a combination thereof.
